(19) United States
(12) Patent Application Publication
(10) Pub. No.: US 2005/0234369 A1
(43) Pub. Date: Oct. 20, 2005

(54) Lockable guide wire tip protector

(75) Inventor: Earl Voorhees, Warrington, PA (US)

Correspondence Address: MONTE & MCGRAW, PC, 4092 SKIPPACK PIKE, P.O. BOX 650, SKIPPACK, PA, 19474, US

(73) Assignee: Medical Components, Inc., Harleysville, PA (US)

(21) Appl. No.: 11/105,029

(22) Filed: Apr. 13, 2005

Related U.S. Application Data
(60) Provisional application No. 60/563,084, filed on Apr. 16, 2004.

(51) Int. Cl. A61M 25/00

(52) U.S. Cl. 600/585; 606/1

(57) ABSTRACT

A guide wire tip protector is disclosed. The protector includes a body having a proximal end and a distal end. The distal end includes a cavity sized to accept a curved distal end of a guide wire and a distal end of a guide wire advancer. The proximal end comprises an elongated portion that releasably engages one of a first guide wire advancer having a generally elongated body and a second guide wire advancer having a ring portion surrounding a generally tubular body. A guide wire assembly incorporating the tip protector is also disclosed.

### CROSS-REFERENCE TO RELATED APPLICATION

The present application claims priority from U.S. Provisional Patent Application Ser. No. 60/563,084, filed 16 Apr. 2004.

### FIELD OF THE INVENTION

The present invention is directed to a guide wire advancer tip protector that is designed to receive and engage one of at least two different designs of guide wire advancers.

### BACKGROUND OF THE INVENTION

Catheters for the introduction or removal of fluids may be located in various venous locations and cavities throughout the body for introduction of fluids to the body or removal of fluids from the body. Such catheterization may be performed by using a single catheter having multiple lumens. A typical example of a multiple lumen catheter is a dual lumen catheter assembly in which one lumen introduces fluid and the other lumen removes fluid. An example of such a dual lumen catheter assembly is the SPLIT-CATH® catheter.

Generally, to insert any catheter into a blood vessel, the vessel is identified by aspiration with a long hollow needle in accordance with the well known Seldinger technique. When blood enters a syringe attached to the needle, indicating that the vessel has been found, a thin guide wire is then introduced, typically through a syringe needle or other introducer device into the interior of the vessel. The introducer device is then removed, leaving the guide wire within the vessel. The guide wire projects beyond the surface of the skin. At this point, several options are available to a physician for catheter placement. The simplest is to pass a catheter into the vessel directly over the guide wire. The guide wire is then removed, leaving the catheter in position within the vessel. However, this technique is only possible in cases where the catheter (for example, a small diameter dual lumen catheter) is of a relatively small diameter, made of a stiff material, and not significantly larger than the guide wire. If the catheter to be inserted is significantly larger than the guide wire, a dilator device is passed over the guide wire to enlarge the opening in the vessel. The catheter is then passed over the guide wire into the vessel, and the guide wire and dilator are then removed, leaving the catheter in position within the vessel.

Typically, the catheter, the syringe, the needle, the guide wire, the dilator, and other necessary equipment are packaged together in a catheter kit. That way, all of the necessary equipment for inserting the catheter is bundled together for easy access by the inserting physician. The guide wire is stored in a guide wire tube to reduce the possibility of kinking the guide wire and of contaminating the guide wire prior to use. However, during shipping, the guide wire occasionally works its way out of the guide wire tube, requiring the inserting physician to re-insert the guide wire into the tube, and potentially contaminating the guide wire prior to use.

Additionally, the distal tip of the guide wire, which is rounded in a “J-shaped” configuration, extends from a distal end of the guide wire tube. The distal tip of the guide wire may be bent or otherwise deformed during shipping, rendering the guide wire unusable.

It would be beneficial to provide a guide wire tip protector to protect the distal tip of the guide wire prior to use and to prevent the guide wire from working its way out of the guide wire tube.

### BRIEF SUMMARY OF THE INVENTION

Briefly, the present invention provides a guide wire tip protector. The tip protector comprises a body having a proximal end and a distal end. The distal end includes a cavity sized to accept a curved distal end of a guide wire and a distal end of a guide wire advancer. The proximal end comprises an elongated portion having means for releasably engaging one of a first guide wire advancer having a generally elongated body and a second guide wire advancer having a ring portion surrounding a generally tubular body.

Additionally, the present invention provides a guide wire tip protector. The tip protector comprises a body having a proximal portion and a distal portion and a longitudinal axis extending therethrough. The distal portion includes an elongated cavity extending therein. The proximal portion includes an elongated member having a generally arcuate concave surface and a plurality of fingers extending therefrom generally toward the longitudinal axis. A first channel is formed between a first and a second of the plurality of fingers on a first side of the longitudinal axis and a second channel is formed between a third and a fourth of the plurality of fingers on a second side of the longitudinal axis.

Further, the present invention provides a guide wire assembly. The assembly comprises a guide wire tube having a distal tube end, a guide wire advancer extending from the distal tube end, and a guide wire having a proximal guide wire end disposed in the tube and a distal guide wire end extending distally of the tube and along a length of the guide wire advancer. The distal guide wire end comprises a distal guide wire tip extending distally of the guide wire advancer. The assembly also includes a guide wire tip protector releasably connected to the guide wire advancer. The distal guide wire tip is disposed within the guide wire tip protector.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a perspective view of a guide wire advancer tip protector according to a preferred embodiment of the present invention.

FIG. 2 is a side elevational view of the guide wire advancer tip protector shown in FIG. 1.

FIG. 3 is a top plan view of the guide wire advancer tip protector shown in FIG. 1.

FIG. 4 is a sectional view of the guide wire advancer tip protector taken along lines 4-4 of FIG. 2.

FIG. 5 is a sectional view of the guide wire advancer tip protector taken along lines 5-5 of FIG. 2.

FIG. 6 is a perspective view of a first design of a guide wire advancer inserted into the guide wire advancer tip protector shown in FIG. 1.

FIG. 7 is a perspective view of a second design of a guide wire advancer inserted into the guide wire advancer tip protector shown in FIG. 1.

### DETAILED DESCRIPTION OF THE INVENTION

In the drawings, like numerals indicate like elements throughout. The words “proximal” and “distal” refer to directions away from and closer to, respectively, the insertion tip of a guide wire in a guide wire advancer used with the guide wire tip protector according to the present invention. The terminology includes the words above specifically mentioned, derivatives thereof, and words of similar import. The following describes a preferred embodiment of the invention. However, it should be understood based on this disclosure, that the invention is not limited by the preferred embodiment described herein.

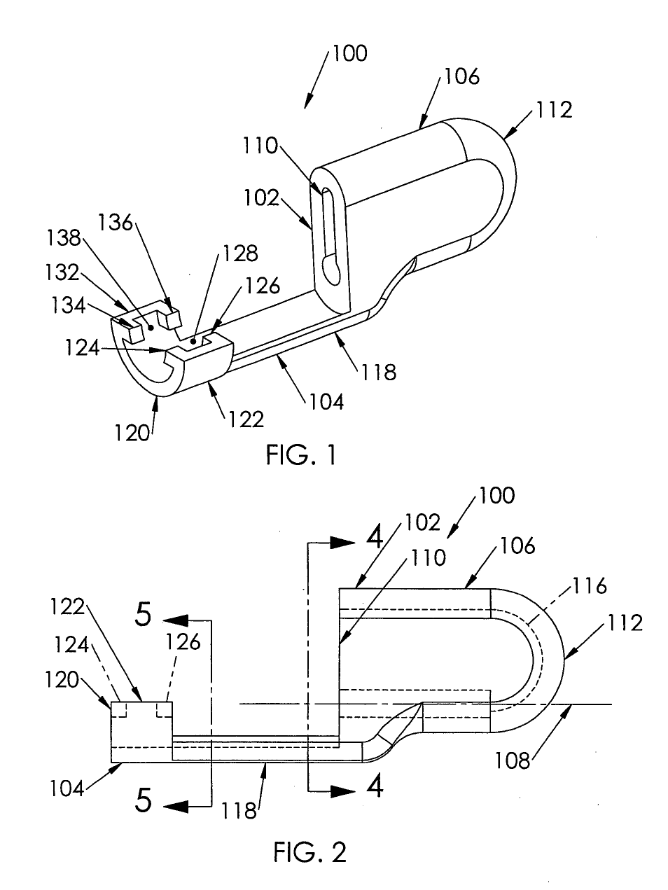

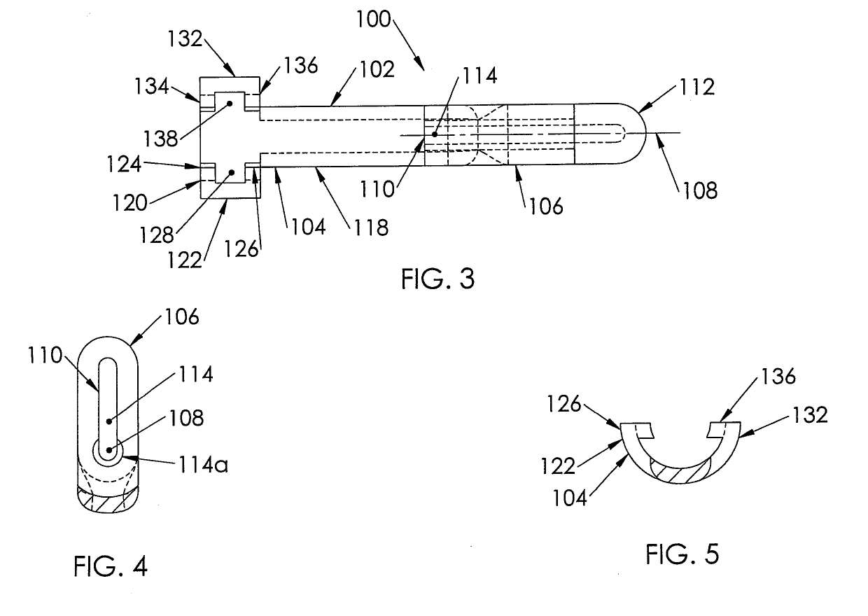

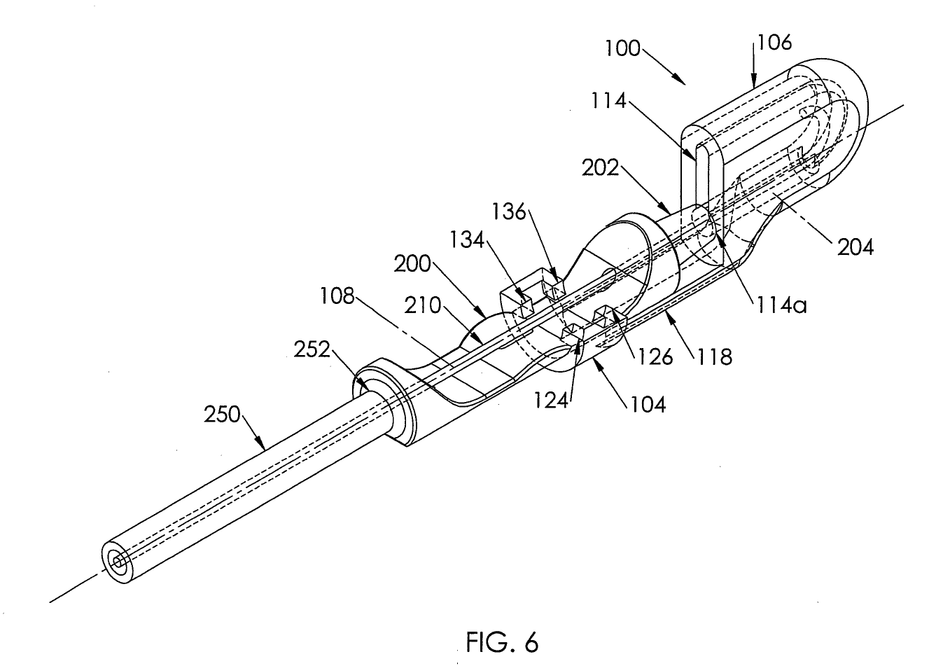

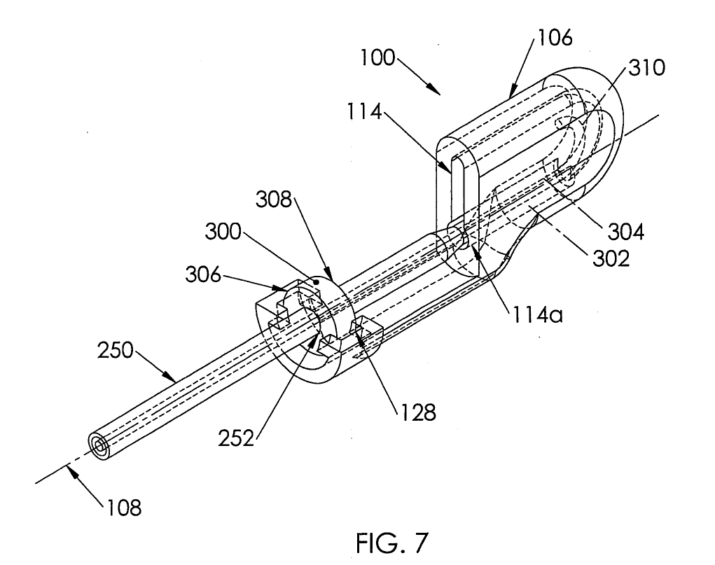

Referring to FIGS. 1-5, a lockable guide wire tip protector 100 according to the present invention is shown. The protector 100 is designed to protect the exposed portion of a guide wire that extends from the distal end of a guide wire advancer assembly. The protector 100 is designed to releasably lockably engage a generally planar guide wire advancer assembly 200 as shown in FIG. 6, or alternatively, to releasably lockably engage a ringed guide wire advancer assembly 300 as shown in FIG. 7.

Referring back to FIGS. 1-5, the protector 100 includes a body 102 having a proximal portion 104, a distal portion 106, and a longitudinal axis 108 extending along the proximal portion 104 and the distal portion 106. The distal portion 106 includes an open end 110 facing the proximal portion 104 and a closed end 112 disposed away from the proximal portion 104. The open end 110 includes a cavity 114 having a generally elongated cross section in a plane perpendicular to the longitudinal axis 108, as shown in FIG. 4. A lower end 114a of the cavity 114 along the longitudinal axis 108 is expanded to a generally circular cross section to accept a distal tip of a guide wire advancer as is also shown in FIG. 4. The cavity 114 ends distally in a preferably arcuate wall 116 that has an approximate radius of curvature as the curved distal tip of a guide wire, as shown in FIG. 2. The closed end 112 of the distal portion 106 is also preferably arcuate.

The proximal portion 104 extends proximally from the open end 110 of the distal portion 106, generally along one side of the longitudinal axis 108. The proximal portion 104 includes a preferably generally elongated body 118, with a generally concave arcuate cross section in a plane perpendicular to the longitudinal axis 108, as shown in FIG. 5. As seen in FIG. 2, a proximal end 120 of the proximal portion 104 that extends on either side of the elongated body 118 ends in a plane containing the longitudinal axis 108.

A first extension 122 extends from the proximal end 120 on one side of the longitudinal axis 108 and includes first finger 124 that extends toward the longitudinal axis 108 and a second finger 126, disposed apart from the first finger 124, that also extends toward the longitudinal axis 108. The first and second fingers 124, 126 form a channel 128 therebetween. A second extension 132 extends from the proximal end 120 on a second side of the longitudinal axis 108 and includes a first finger 134 that extends toward the longitudinal axis 108 and a second finger 136, disposed apart from the first finger 134, that also extends toward the longitudinal axis 108. The first and second fingers 134, 136 form a channel 138 therebetween.

The protector 100 is preferably constructed from a polymer, and more preferably, from a generally transparent or translucent polymer, such as a thermoplastic elastomer sold under the mark DYNAFLEX®, sold by GLS Corp. of McHenry, Ill. or KRATON®, sold by Shell Oil Company of Houston Tex. Also preferably, the protector 100 has a hardness of approximately 82A on the Shore Durometer scale. The protector 100 may be manufactured by injection molding, or by any suitable methods, as will be recognized by those skilled in the art.

The protector 100 is sized to releasably retain one of at least two different designs of guide wire advancers that are presently on the market. The first type of advancer 200, shown in FIG. 6, includes a conical distal tip 202 that is inserted into the lower end 114a of the cavity 114 in the distal portion 106 of the protector 100. The distal tip 202 preferably engages the walls of the lower end 114a of the cavity 114 in a generally interference fit around the proximal end of the cavity 114. A guide wire 210, extending through the advancer 200, that extends in a curved fashion from the distal end 204 of the conical distal tip 202, is inserted into the cavity 114.

The advancer 200 includes a generally elongated body that extends along the generally elongated body 118 of the proximal portion 104, such that the generally elongated body of the advancer 200 is disposed generally between the generally elongated body 118 of the proximal portion 104 and the longitudinal axis 108. The fingers 124, 126, 134, 136 retain the advancer 200 against the proximal portion 104 of the protector 100 so that the protector 100 does not disengage from the advancer 200. When the advancer 200 is securely connected to the protector 100, the guide wire 210 that extends through the advancer 200 also extends along the longitudinal axis 108.

The advancer 200 is connected to a guide wire tube 250. The guide wire tube 250 includes a distal end 252 that is connected to the advancer 200. Preferably, the advancer 200 is removable from the distal end 252 of the guide wire tube 250, although those skilled in the art will recognize that the advancer 200 may be fixedly connected to the distal end 252 of the guide wire tube 250.

Prior to using the advancer 200, the protector 100 is removed from the advancer 200 by rotating the advancer 200 about the longitudinal axis 108 relative to the protector 100 to disengage the fingers 124, 126, 134, 136 from the elongated body of the advancer 200 and then by sliding the advancer 200 generally along the longitudinal axis 108 proximally relative to the protector 100.

The second advancer 300, shown in FIG. 7, includes a conical distal tip 302 that is inserted into the lower end 114a of the cavity 114 in the distal portion 106 of the protector 100. The distal tip 302 preferably engages the walls of the lower end 114a of the cavity 114 in a generally interference fit around the proximal end of the cavity 114. A guide wire 310, extending through the advancer 300, that extends in a curved fashion from the distal end 304 of the conical distal tip 302, is inserted into the cavity 114. A proximal end 306 of the advancer 300 includes a ring 308 that extends around the body of the advancer 300. A portion of the ring 308 is inserted into the channels 128, 138 in a preferably interference fit so that the protector 100 does not disengage from the advancer 300. When the advancer 300 is securely connected to the protector 100, the guide wire 310 that extends through the advancer 300 also extends along the longitudinal axis 108.

The advancer 300 is connected to the distal end 252 of the guide wire tube 250. Preferably, the advancer 300 is removable from the distal end 252 of the guide wire tube 250 so that the guide wire 310 may be advanced distally from the advancer 300.

Prior to using the advancer 300, the protector 100 is removed from the advancer 300 by removing the ring 308 from the channels 128, 138 and then by sliding the advancer 300 generally along the longitudinal axis 108 proximally relative to the protector 100.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A guide wire tip protector comprising a body having a proximal end and a distal end, wherein the distal end includes a cavity sized to accepted a curved distal end of a guide wire and a distal end of a guide wire advancer, and wherein the proximal end comprises an elongated portion having means for releasably engaging one of a first guide wire advancer having a generally elongated body and a second guide wire advancer having a ring portion surrounding a generally tubular body.

2. The guide wire tip protector according to claim 1, further comprising an elongated body extending between the proximal end and the distal end.

3. The guide wire tip protector according to claim 2, wherein the elongated body comprises a generally concave cross section.

4. The guide wire tip protector according to claim 1, wherein the cavity comprises an arcuate distal end.

5. The guide wire tip protector according to claim 1, wherein the cavity comprises an expanded portion sized to accept the distal end of the guide wire advancer.

6. A guide wire tip protector comprising a body having a proximal portion and a distal portion and a longitudinal axis extending therethrough, wherein the distal portion includes an elongated cavity extending therein and wherein the proximal portion includes an elongated member having a generally arcuate concave surface and a plurality of fingers extending therefrom generally toward the longitudinal axis, wherein a first channel is formed between a first and a second of the plurality of fingers on a first side of the longitudinal axis and a second channel is formed between a third and a fourth of the plurality of fingers on a second side of the longitudinal axis.

7. The guide wire tip protector according to claim 6, further comprising an elongated body extending between the proximal portion and the distal portion.

8. The guide wire tip protector according to claim 7, wherein the elongated body comprises a generally concave cross section.

9. The guide wire tip protector according to claim 6, wherein the cavity comprises an arcuate distal end.

10. The guide wire tip protector according to claim 6, wherein the cavity comprises an expanded portion sized to accept the distal end of the guide wire advancer.

11. A guide wire assembly comprising:
a guide wire tube having a distal tube end;
a guide wire advancer extending from the distal tube end;
a guide wire having a proximal guide wire end disposed in the tube and a distal guide wire end extending distally of the tube and along a length of the guide wire advancer, wherein the distal guide wire end comprises a distal guide wire tip extending distally of the guide wire advancer; and
a guide wire tip protector releasably connected to the guide wire advancer, wherein the distal guide wire tip is disposed within the guide wire tip protector.

12. The guide wire assembly according to claim 11, wherein the guide wire advancer comprises one of a first guide wire advancer having a generally elongated body and a second guide wire advancer having a ring portion surrounding a generally tubular body.

13. The guide wire assembly according to claim 11, wherein the guide wire tip protector comprises a body having a proximal end and a distal end, wherein the distal end includes a cavity sized to accepted a curved distal end of a guide wire and a distal end of a guide wire advancer, and wherein the proximal end comprises an elongated portion having means for releasably engaging one of a first guide wire advancer having a generally elongated body and a second guide wire advancer having a ring portion surrounding a generally tubular body.

14. The guide wire tip protector according to claim 13, further comprising an elongated body extending between the proximal end and the distal end.

15. The guide wire tip protector according to claim 14, wherein the elongated body comprises a generally concave cross section.

16. The guide wire tip protector according to claim 13, wherein the cavity comprises an arcuate distal end.

17. The guide wire tip protector according to claim 13, wherein the cavity comprises an expanded portion sized to accept the distal end of the guide wire advancer.
